(19) United States
(12) Patent Application Publication
(10) Pub. No.: US 2025/0346959 A1
(43) Pub. Date: Nov. 13, 2025

(54) DETECTION OF MODIFIED NUCLEOBASES IN DNA SAMPLES

(71) Applicant: Roche Sequencing Solutions, Inc., Pleasanton, CA (US)

(72) Inventors: Jagadeeswaran Chandrasekar, Seattle, WA (US); Joseph W. Horsman, Seattle, WA (US); Mark Stamatios Kokoris, Bothel, WA (US); Robert N. McRuer, Mercer Island, WA (US); Marc Prindle, Seattle, WA (US); John C. Tabone, Kirkland, WA (US)

(21) Appl. No.: 19/266,880

(22) Filed: Jul. 11, 2025

Related U.S. Application Data
(63) Continuation of application No. PCT/EP2024/050590, filed on Jan. 11, 2024.
(60) Provisional application No. 63/479,959, filed on Jan. 13, 2023.

(51) Int. Cl. C12Q 1/6886; C12Q 1/683

(52) U.S. Cl. CPC C12Q 1/6886; C12Q 1/683; C12Q 2600/154

(57) ABSTRACT

Described are methods of detecting modified nucleotide bases in a DNA sample using specific DNA glycosylases to excise a modified nucleobase of interest. Prior to glycosylase treatment, DNA target fragments are copied by a DNA polymerase to produce a complementary copy strand that preserves the genetic information of the DNA target strand. Following glycosylase treatment, the DNA target fragments are repaired by either ligating across the gaps to produce a deletion at each position of the modified nucleobase of interest or filling in the gaps with a single non-native nucleotide to produce a base substitution at each position of the modified nucleobase of interest. Comparison of the DNA sequences of the two strands of the target fragments enables identification of the positions of the modified nucleotide base in the DNA target fragment.

### BACKGROUND

Methylation and the products of various forms of DNA damage have been implicated in a variety of important biological processes. Changes in methylation patterns and the appearance of damaged DNA are often among the earliest events observed for various disease states

Epigenetic modifications are essential for normal development. For example, methylcytosine, the most widely studied epigenetic modification, is associated with a number of key processes including genomic imprinting, X-chromosome inactivation, suppression of repetitive elements, and carcinogenesis. For example, DNA methylation at the 5 position of cytosine has the specific effect of reducing gene expression and has been found in every vertebrate examined. In many disease processes, such as cancer, gene promoter CpG islands acquire abnormal hypermethylation, which results in transcriptional silencing that can be inherited by daughter cells following cell division. In addition, alterations of DNA methylation have been recognized as an important component of cancer development. Hypomethylation, in general, arises earlier and is linked to chromosomal instability and loss of imprinting, whereas hypermethylation is associated with promoters and can arise secondary to gene (oncogene suppressor) silencing. Additionally, hydroxymethylcytosine has also emerged as an important epigenetic modification as well with potential regulatory roles in gene expression ranging from development to aging. Various cancers have shown that hydroxymethylcytosine content is consistently and significantly reduced in malignant versus healthy tissues, even in early-stage lesions.

DNA is under constant stress from both endogenous and exogenous sources. The bases exhibit limited chemical stability and are vulnerable to chemical modifications through different types of damage, including oxidation, alkylation, radiation damage, and hydrolysis. Damage to DNA bases may affect their base-pairing properties and, therefore, may be mutagenic. DNA base modifications resulting from these types of DNA damage are wide-spread and play important roles in affecting physiological states and disease phenotypes. Examples include 7,8-dihydro-8-oxoguanine (8-oxoG) (oxidative damage), 8-oxoadenine (oxidative damage; aging, Alzheimer's, Parkinson's), 1-methyladenine, 06-methylguanine (alkylation; gliomas and colorectal carcinomas), benzo[a]pyrene diol epoxide (BPDE), pyrimidine dimers (adduct formation; smoking, industrial chemical exposure, UV light exposure; lung and skin cancer), and 5-hydroxy cytosine, 5-hydroxyuracil, 5-hydroxymethyluracil, and thymine glycol (ionizing radiation damage; chronic inflammatory diseases, prostate, breast and colorectal cancer). For example, 8-oxoG is a frequent product of DNA oxidation. 8-oxoG tends to base-pair with adenine, giving rise to G»C to T·A transversion mutations. Another example is the hydrolytic deamination of cytosine and 5-methylcytosine (5-meC) to give rise to uracil and thymine mispaired with guanine, respectively, causing C»G to T·A transition mutations if not repaired. In another example, alkylation can generate a variety of DNA base lesions comprising 6-meG, N7-methylguanine (7-meG), or N3-methyladenine (3-meA). While 6-meG is promutagenic by its property to pair with thymine, 7-meG and 3-meA block replicative DNA polymerases and are therefore cytotoxic. These and many other forms of DNA base damage arise in cells many times every day and only the continuous action of specialized DNA repair systems can prevent a rapid decay of genetic information. In addition to damage to nuclear DNA, mitochondrial DNA also experience significant oxidative damage, as well as damage from alkylation, hydrolysis, and adducts. For example, oxidative damage is the most prevalent type of damage in mitochondrial DNA, primarily because mitochondria are a major cellular source of reactive oxygen species (ROS). In addition, mitochondria house approximately 30% of the cellular pool of S-adenosylmethionine, which can methylate DNA nonenzymatically. Also, exposure to certain agents, such as estrogens, tobacco smoke, and certain chemicals, leads to preferential damage of mitochondrial DNA.

As DNA damage and epigenetic modification may be the earliest indications of disease state, detection of epigenetic modification and DNA damage patterns can be useful for early detection of disease and intervention. However, detection methods have limitations. For example, with respect to methylation status, spectrophotometry can be used to indicate global content of a modification in target DNA, but has limited specificity. High-performance liquid chromatography (HPLC) and mass spectrometry are also often used, but are costly, require significant amounts of material, and reduce DNA to constituent nucleosides or nucleotides, thus destroying sequence information for downstream analysis. Immunoprecipitation (IP) using monoclonal antibodies can enrich DNA with target modifications, but limitations with specificity have been identified. Restriction digest profiling utilizes fragment analysis of DNA treated with modification-sensitive restriction endonucleases, but requires large amounts of material and is limited to sequences featuring a restriction site with known sensitivity. While bisulfite sequencing is considered the “gold-standard” technique for detection of DNA methylation, there are important limitations. First, the chemical conversion process causes widespread non-specific damage to DNA, and thus the approach requires large amounts of starting material. Second, the method can be expensive and time consuming, requiring multiple sequencing runs. Finally, and importantly, it is generally only applicable to methylcytosine (mC) modifications. Variations have been developed or suggested that allow a limited number of additional modification types to be targeted (methylcytosine (mC) and hydroxymethylcytosine (hmC)) but these are low-yield and still share the other limitations listed above. They are also not readily applicable to other modifications and are fairly complex.

Thus, there is a need in the art for improved methods of detecting modified nucleobases in DNA samples of interest. The present invention fulfills these needs and provides further related advantages as discussed below.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### BRIEF SUMMARY OF THE INVENTION

Aspects of the present invention encompass detection of modified nucleobases, such as epigenetic changes and DNA damage, in DNA samples.

In one aspect, the invention provides a method of detecting a modified nucleobase in a plurality of nucleic acids, the method including: providing a sample including a plurality of DNA templates; generating complementary copies of the DNA templates, the generating being directed by an oligonucleotide primer using a DNA polymerase in the presence of native dNTPs, in which the generating produces a complementary copy of each of the DNA templates such that each complementary copy is hybridized to one of the DNA templates; subjecting the DNA templates and the complementary copies to a base excision repair enzyme treatment, in which the base excision repair enzyme specifically excises the nucleotides comprising the modified nucleobase from the DNA templates to produce a single stranded gap at the positions of the modified nucleobase, and in which the complementary copies are resistant to treatment with the base excision repair enzyme; repairing the single stranded gaps in the DNA templates to produce contiguous DNA template strands; determining the nucleotide sequences of the contiguous DNA template strands and the complementary copies; and comparing the nucleotide sequences of contiguous DNA template strands and the complementary copies, thereby determining the positions of the modified nucleobase in the DNA templates prior to base excision repair enzyme treatment.

In one embodiment, the step of repairing the gaps to form the contiguous full length DNA target fragment strands includes the step of treating the double stranded DNA fragments with a DNA ligase enzyme, thereby producing deletions in the DNA target fragment strands at each of the positions of the nucleotides comprising the modified nucleobase of interest. In some embodiments, the DNA ligase enzyme is T4 DNA ligase.

In another embodiment, the step of repairing the gaps to form the contiguous full length DNA target fragments includes the step of treating the double stranded DNA fragment strands with a DNA polymerase in the presence of a non-native nucleotide and a DNA ligase, thereby producing a nucleotide substitution in the DNA target fragment strands at each of the positions of the nucleotides comprising the modified nucleobase of interest. In one embodiment, the DNA polymerase does not exhibit exonuclease or strand displacing activity and the DNA ligase enzyme is not capable of ligating across single stranded gaps. In yet another embodiment, the DNA polymerase is Klenow exo- or T4 DNA polymerase and the DNA ligase is e. coli DNA ligase.

In some embodiments, the step of comparing the nucleotide sequences of the DNA target fragments and the complementary copy strands identifies one or more differences in the sequence of the DNA target fragments strands relative to the sequence of the_complementary copy strands, in which the positions of the one or more differences identifies the positions of the modified nucleobase base of interest in the DNA target fragments. In certain embodiments, the one or more differences in the sequence of the DNA target fragments strands relative to the sequence of the_complementary copy strands are one or more mutations, one or more deletions, or one or more substitutions.

In some embodiments, the base excision repair enzyme is selected from the group of enzymes set forth in Table 1.

In some embodiments, the base excision repair enzyme is selected from N-methylpurine DNA Glycosylase (MPG), MutY Homolog (MUTYH), Nth-like DNA Glycosylase 1 (NTHL1), Nei-like DNA Glycosylase 1 (NEIL1), Nei-like DNA Glycosylase 2 (NEIL2), Nei-like DNA Glycosylase 3 (NEIL3), 8-oxoguanine DNA glycosylase (OGG1), Uracil DNA Glycosylase 1 (Ung1), Uracil DNA Glycosylase 2 (Ung2), Single-strand selective monofunctional uracil glycosylase (SMUG1), Thymine DNA Glycosylase (TDG), Methyl binding domain 4 (MBD4), FPG, Ung, Demeter (DME), DEMETER-like protein 2 (DMEL-2), DEMETER-like protein 3 (DMEL-3), ROS1, UDG, Apurinic endonuclease (APE1), DNA polymerase beta (POLB), XRCC1, DNA Ligase 1 (LIG1), DNA Ligase 3 (LIG3), and DNA polymerase gamma (POLG). In certain embodiments, the base excision repair enzyme includes a multifunctional DNA glycosylase enzyme, in which the multifunctional DNA glycosylase enzyme exhibits both glycosylase activity and lyase activity. In some embodiments, the multifunctional DNA glycosylase enzyme is FPG, DME, ROS1, DMEL-2, or DMEL-3. In other embodiments, the base excision repair enzyme includes a first enzyme exhibiting glycosylase activity and a second enzyme exhibiting lyase activity. In one embodiment, the first enzyme is TDG or UDG and the second enzyme is FPG, DME, ROS1, DMEL-2, or DMEL-3.

In some embodiments, the DNA polymerase is a high-fidelity DNA polymerase.

In some embodiments, the double stranded DNA target fragments are genomic DNA, mitochondrial DNA, cell-free DNA, circulating tumor DNA, or combinations thereof.

In some embodiments, the modified base of interest is 5-mC, 5-hmC, 5-fC, and/or 5-caC.

In one embodiment, the single stranded adaptor-ligated DNA target fragments are immobilized on a solid support. In another embodiment, the complementary copy strands are immobilized on a solid support.

In some embodiments, the method further includes the step of polishing the single stranded gaps with one or more enzymes to produce a free 3′ hydroxyl and a free 5′ phosphate group at the positions of each of the gaps. In one embodiment, the one or more enzymes includes APE1, Endonuclease B, PolB, and/or PNK.

In some embodiments, the non-native nucleotide is dZTP, dPTP, dSTP, or dBTP.

In some embodiments, the DNA templates include a first adapter joined to the 5′ end of the DNA template and a second adapter joined to the 3′ end of the DNA template. In certain embodiments, the first adapter is a Y adapter and the second adapter is a Y adapter or a hairpin adapter. In some embodiments, at least one of the first and the second adapters includes a unique molecular identifier barcode (UMI). In further embodiments, the step of comparing the sequences of the contiguous DNA template strands and the complementary copies includes bioinformatically pairing the sequences comprising the same unique molecular barcode (UMI).

### BRIEF DESCRIPTION OF THE DRAWINGS

FIG. 1 is a condensed schematic summarizing alternative embodiments of the methods of the present invention.

FIG. 2 is a condensed schematic summarizing alternative embodiments of the methods of the present invention.

FIG. 3 depicts the structures of two embodiments of non-natural nucleobases.

FIGS. 4A and 4B are cartoons summarizing one embodiment of a work-flow for generating gaps in a DNA target fragments at the positions of a modified nucleobase of interest and subsequent detection steps.

FIGS. 5A and 5B are schematics illustrating alternative embodiments of solid-state synthesis of primer extension reactions.

FIG. 6 depicts the generalized structure of an XNTP.

### DETAILED DESCRIPTION OF THE INVENTION

The present invention may be understood more readily by reference to the following detailed description of preferred embodiments of the invention and the Examples included herein. Unless otherwise explained, all technical and scientific terms used herein have the same meaning as commonly understood by one of ordinary skill in the art to which this disclosure belongs.

Reference throughout this specification to “one embodiment” or “an embodiment” and variations thereof means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used in this specification and the appended claims, the singular forms “a,” “an,” and “the” include plural referents, i.e., one or more, unless the content and context clearly dictates otherwise. It should also be noted that the conjunctive terms, “and” and “or” are generally employed in the broadest sense to include “and/or” unless the content and context clearly dictates inclusivity or exclusivity as the case may be. Thus, the use of the alternative (e.g., “or”) should be understood to mean either one, both, or any combination thereof of the alternatives. In addition, the composition of “and” and “or” when recited herein as “and/or” is intended to encompass an embodiment that includes all of the associated items or ideas and one or more other alternative embodiments that include fewer than all of the associated items or ideas.

Unless the context requires otherwise, throughout the specification and claims that follow, the word “comprise” and synonyms and variants thereof such as “have” and “include”, as well as variations thereof such as “comprises” and “comprising” are to be construed in an open, inclusive sense, e.g., “including, but not limited to.” The term “consisting essentially of” limits the scope of a claim to the specified materials or steps, or to those that do not materially affect the basic and novel characteristics of the claimed invention.

The abbreviation, “e.g.,” is derived from the Latin exempli gratia, and is used herein to indicate a non-limiting example. Thus, the abbreviation “e.g.,” is synonymous with the term “for example.” It is also to be understood that as used herein and in the appended claims, the singular forms “a,” “an,” and “the” include plural reference unless the context clearly dictates otherwise, the term “X and/or Y” means “X” or “Y” or both “X” and “Y”, and the letter “s” following a noun designates both the plural and singular forms of that noun. In addition, where features or aspects of the invention are described in terms of Markush groups, it is intended, and those skilled in the art will recognize, that the invention embraces and is also thereby described in terms of any individual member and any subgroup of members of the Markush group, and Applicants reserve the right to revise the application or claims to refer specifically to any individual member or any subgroup of members of the Markush group.

Any headings used within this document are only being utilized to expedite its review by the reader, and should not be construed as limiting the invention or claims in any manner. Thus, the headings and Abstract of the Disclosure provided herein are for convenience only and do not interpret the scope or meaning of the embodiments.

Where a range of values is provided herein, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either or both of those included limits are also included in the invention.

For example, any concentration range, percentage range, ratio range, or integer range provided herein is to be understood to include the value of any integer within the recited range and, when appropriate, fractions thereof (such as one tenth and one hundredth of an integer), unless otherwise indicated. Also, any number range recited herein relating to any physical feature, such as polymer subunits, size or thickness, are to be understood to include any integer within the recited range, unless otherwise indicated. As used herein, the term “about” means±20% of the indicated range, value, or structure, unless otherwise indicated.

### Methods of Detecting Modified DNA Nucleobases

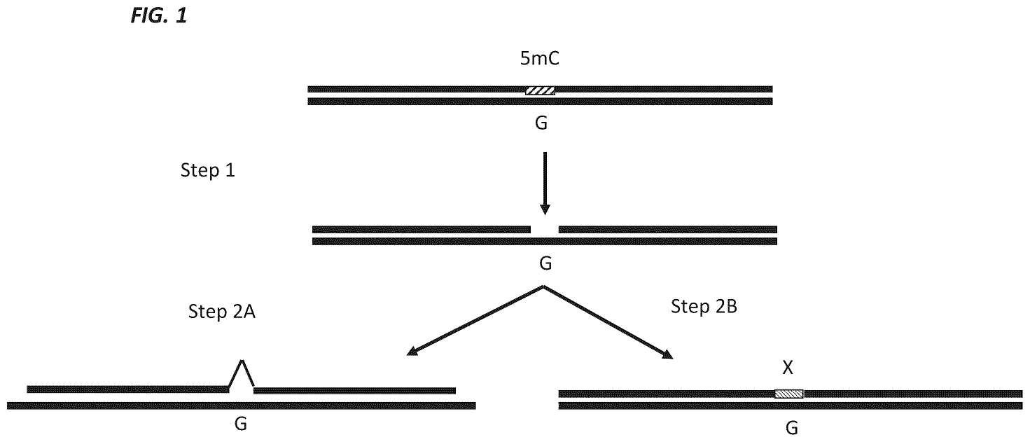

Described herein are alternative general methods of determining the location and identity of modified DNA nucleobases, such as those arising from epigenetic modifications or DNA damage, in DNA target fragment templates. These methods are outlined in FIG. 1, wherein the modified nucleobase of interest, in this embodiment, is 5-methylcytosine (5-mC). As depicted in this exemplary embodiment, the top strand of the double stranded DNA target fragment includes a single 5-mC residue (represented by the hatched portion of the top strand) base paired with G, while the bottom strand does not include 5-mC residues. Both methods are based on the specific excision of the nucleotides comprising the modified nucleobase of interest (i.e., nucleotides of interest), thereby creating a single stranded gap at each of the positions of the modified nucleotides of interest (“Step 1” depicted in FIG. 1). The identity of the modified nucleobase is determined by the enzyme or chemistry used to specifically excise the nucleotides comprising the modified nucleobase. Following excision of the nucleotides of interest, the locations of the resulting single stranded gaps can be assessed by two alternative methods for repairing the gaps created in Step 1. The first method includes the step of ligating across the gaps to create a contiguous DNA template strand with a deletion at each position of the gaps created in Step 1. (“gap ligation”, see “Step 2A”, depicted in FIG. 1). The second method includes the step of filling in the gaps with a nucleotide comprising an alternative, e.g., non-natural, DNA base to produce a contiguous DNA target strand (“gap fill”, see “Step 2B” depicted in FIG. 1).

These methods offer improvements over state-of-the-art workflows for epigenetic detection based, e.g., on bisulfite conversion, by technical shortcomings well known in the art, such as DNA degradation and reduced genomic complexity. The methods disclosed herein are based on enzymatic excision of modified nucleotides of interest and are thus more rapid and specific, less degradative, and do not require specialized reagents. Moreover, the modified (i.e., “converted”) DNA templates are stable and readily amplified by, e.g., PCR.

As discussed above, the methods disclosed herein include enzymatic excision of nucleotides comprising a modified base of interest in double stranded DNA target fragment templates to produce a single gap at each position in which the nucleotides of interest occurs in the nucleic acid sequence of the DNA templates. The single stranded gaps are subsequently repaired to produce contiguous DNA template strands, either by a gap-ligation or a gap-fill process. The positions of the repaired gaps can be identified by multiple DNA sequencing methodologies, as described herein.

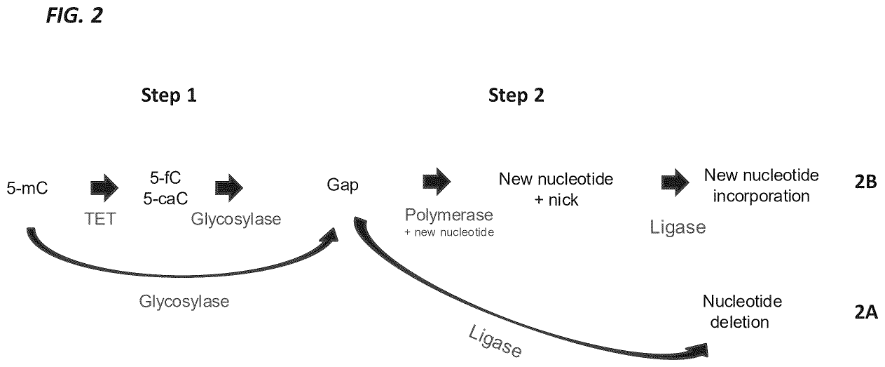

Further details of the methods disclosed herein are illustrated in FIG. 2. As discussed, both methods share an upstream workflow (“Step 1” depicted in FIG. 2) that includes generating single stranded gaps by specific excision of the nucleotides comprising the modified base of interest. The specificity of the excision ensures that the nucleobase of interest can be detected by identifying the locations of the newly created gaps.

One method of specifically generating single stranded, single nucleotide gaps in DNA target fragments is by utilizing DNA glycosylases, a family of enzymes that are also referred to in the art as “base excision repair” enzymes. The diversity of DNA glycosylases allows for many different nucleobase modifications to be assayed by the methods disclosed herein. Enzymes that excise only the nucleobase, yielding an abasic site, can also be utilized, as abasic sites can be further reacted to form single nucleotide gaps.

In the embodiment depicted in FIG. 2, epigenetic methylation of cytosine (e.g., 5-mC) may be assayed by subjecting a DNA target fragment to treatment with a member of the Demeter/ROS1 family of glycosylases, which act directly on 5-mC by excising the nucleotide comprising this epigenetic mark. Alternatively, 5-mC may be first converted to 5-formylcytosine (5-fC) or 5-carboxylcytosine (5-caC), via oxidation mediated by the ten-eleven translocation (TET) methylcytosine dioxygenases. 5-fC and 5-caC can then be specifically excised by, e.g., thymine DNA glycosylase (TDG).

One method to repair the gaps formed in Step 1 is via “gap-ligation”, as illustrated in Step 2A (“2A” depicted in FIG. 2). In certain embodiments, this method leverages the ability of T4 DNA ligase to ligate across small, single stranded gaps in otherwise contiguous double stranded DNA. This cross-gap ligation produces double stranded DNA with a “bulged” base opposite the ligation site (i.e., a deletion in the strand comprising the targeted modified nucleobase and an intact opposite strand). Since the gaps are repaired to produce contiguous DNA template strands, both strands of a double stranded target fragment can be amplified in a PCR reaction. When sequenced, the gap-ligated DNA strand will be read as containing a deletion at each gap site when compared to a reference sequence.

When employing to this method, it is advantageous to utilize a sequencing technology with low deletion error rates in order to minimize sequencing errors that could generate false signals. There are several methods known in the art to reduce type 1 (false positive) errors. Additional information, such as sequence context, identity of the deleted base, and generation of multiple sequence reads can identify excised modified bases with high certainty. As the chemistry used to excise the nucleotides of interest is controlled, the method offers a priori knowledge of which of the four DNA bases is being detected. Sequence context, such as CpG for DNA methylation, can further validate that deletions detected are not sequencing errors. As the method is compatible with PCR amplification, unique molecular identifiers (UMI) can be leveraged to provide consensus sequences prior to identifying gap-ligation events.

An alternative method to repair the gaps formed in Step 1 is via “gap-fill”, as illustrated in Step 2B (“2B” depicted in FIG. 2). In this embodiment, a nucleotide comprising an alternative, e.g., non-native or non-standard, nucleobase can be incorporated into the gaps by a DNA polymerase. The polymerase incorporation, i.e., gap fill, leaves a nick in the DNA backbone that can subsequently be sealed by a DNA ligase. By providing the DNA polymerase with a single non-native nucleotide, even weak polymerase incorporation can result in fill-in of the single nucleotide gaps. Examples of non-standard nucleotides are disclosed in, e.g., U.S. Pat. No. 9,334,534, which is hereby incorporated by reference in its entirety.

The selection of enzymes for the gap-fill protocol is important to ensure that the method is specific for incorporating and ligating non-native nucleotides without disrupting the DNA template comprising nucleotide gaps or nicks in the backbone. Preferred DNA polymerases are those lacking robust exonuclease/proofreading or strand displacement activities. In certain embodiments, a suitable DNA polymerase may be Klenow exo- or T4 DNA polymerase. In other embodiments, a suitable DNA ligase is one that cannot ligate across DNA gaps, e.g., E. coli DNA ligase.

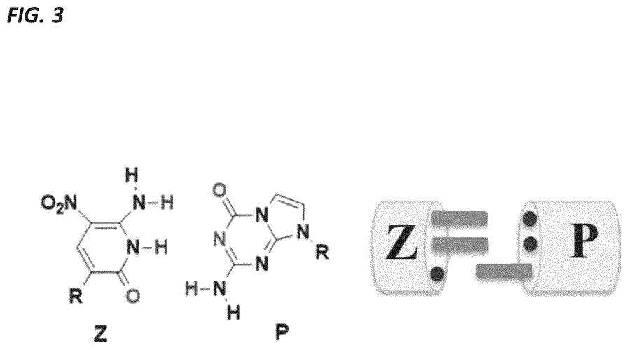

In certain embodiments, PCR can be utilized to amplify DNA template strands that have been repaired with nucleotides comprising non-native bases (as used herein, the terms “non-native”, “non-natural”, and “non-standard” are used interchangeably). For example, the methods may employ two non-native bases that specifically and accurately base pair, thus increasing the “DNA alphabet” from 4 bases to 6 bases. One non-native base is incorporated into the DNA target fragment during the gap-fill repair process of Step 2B in FIG. 2, while the pair of non-native bases is incorporated during subsequent PCR amplification of the repaired DNA target fragments. One exemplary pair of non-native bases that may be used according to the present invention is dZTP and dPTP (available, e.g., from Firebird Biomolecular Sciences, ltd.), which are illustrated in FIG. 3.

In certain embodiments, DNA template strands repaired by gap-fill with non-native bases can be sequenced directly by modifying existing DNA sequencing technologies to include a reagent that specifically base pairs with the non-native base. For example, in certain embodiments, a fluorescent nucleoside that pairs with the non-native base enables detection with optical sequencing-by-synthesis methods. In other embodiments, an expandable nucleotide that base pairs with the non-native base could enable sequencing-by-expansion to directly detect the non-native base.

In some embodiments, the methods disclosed herein may also include additional steps. For example, the methods may include a step to repair, or “polish”, the DNA target fragments prior to Step 1 outlined in FIGS. 1 and 2. Such treatment may ensure that there is no pre-existing damage in the DNA target fragment, e.g., strand nicks, breaks and the like, that could lead to false positive errors in downstream analysis.

In other embodiments, the DNA target fragments may optionally be modified to facilitate excision of specific DNA nucleobases. As described herein, in one embodiment, specific DNA nucleobases may be oxidized, e.g., with ten-eleven translocation (TET) methylcytosine dioxygenases, which oxidize 5-mC to 5f-C and 5-caC. In other embodiments, 8-oxo-G damage may be specifically excised by DNA-formamidopyrimidine glycosylase.

In other embodiments, the methods may include a “polishing” step following Step 1 and prior to Step 2. It is known in the art that DNA glycosylases can create a variety of functional groups post-cleavage or excision of the target modified nucleobase. Prior to repair of the single stranded gaps, the ends 5′ and 3′ of the gaps must treated to provide the correct chemical moieties (e.g., a 5′ hydroxy group and a 3′ phosphate group) for gap-ligation or gap-fill. In one embodiment, treatment of the gaps in DNA target fragments with polynucleotide kinase (PNK) can generate the necessary 5′ and 3′ functional groups. In other embodiments, the polishing step can include treatment with a cocktail of DNA repair enzymes, e.g., a mixture of APE, a phosphatase, and a kinase.

In certain embodiments, both the gap-ligation and gap-fill reactions can be combined into a single, multi-enzyme reaction, both for the purposes of simplicity and to reduce reaction times and potential sources of error. In one embodiment, the four steps of: 1) modified nucleotide excision; 2) gap-fill; 3) end polishing; and 4) ligation can be combined. This approach minimizes the lifetime of the single nucleotide gaps, which are potentially unstable, leading to double stranded breaks. By combining these four steps into a “one pot” reaction, the various reactions may proceed rapidly through unstable intermediates and yield stable, contiguous DNA template strands.

In certain embodiments, the methods of the present invention also include a workflow that generates a complementary copy (i.e., a “daughter” strand) of the DNA template (i.e., the “parent” strand). Importantly, the complementary copy is generated before the step of enzymatic excision of the nucleotides comprising the modified nucleobases of interest. The daughter strand thus encodes the genetic information of the DNA template, and thereby functions as a reference sequence, while the parent strand, through enzymatic conversion, encodes the epigenetic information. Sequence information obtained from the complementary copy and template strands can be paired bioinformatically and compared to identify the positions of the modified nucleobase of interest in the nucleic acid sequence of the original DNA target fragment.

Overview of “Parent-Daughter” Library Workflow

For each of the methods described herein, the modified nucleobase of interest may be at least one of 5-methylcytosine (5-mC), 5-hydroxymethylcytosine (5-hmC), 5-carboxycytosine (5-caC), 5-formylcytosine (5-fC), 8-oxo-7,8-dihyroguanine (*-oxoG), uracil, 6-methyladenine (6-mA), or 8-oxoadenine, O-6-methylguanine, 1-methyladenine, O-4-methylthymine, 5-hydroxy cytosine, 5-hydroxyuracil, 5-hydroxymethyluracil, or thymine dimers. In some instances, a plurality of any combination of these types of modified nucleobases may be detected.

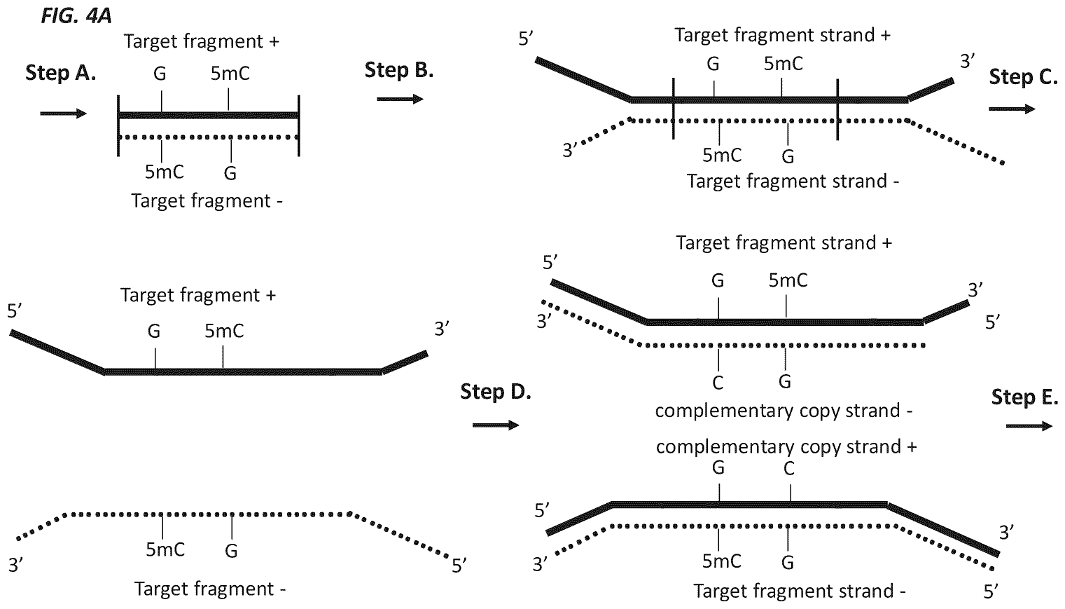

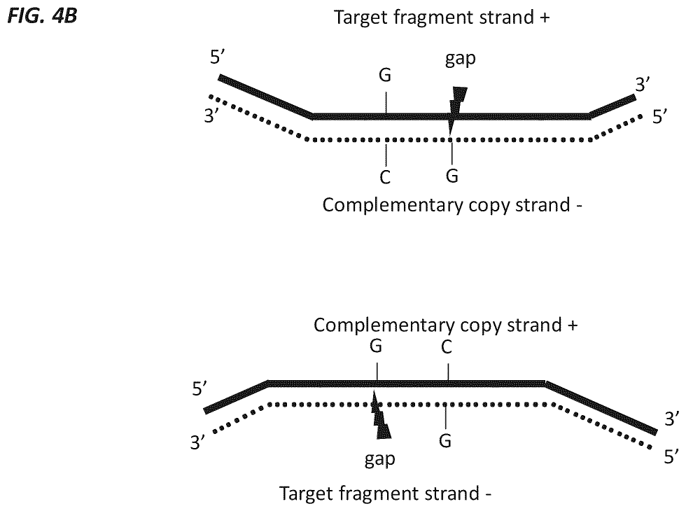

In one aspect, provided are methods for detecting a modified DNA nucleobase in a DNA sample. An exemplary schematic overview of the methods is provided in FIGS. 4A and 4B. The methods may include obtaining a DNA sample and fragmenting the DNA to produce a sample of DNA target fragments (Step A). As used herein, the term “target fragment” means that the corresponding DNA fragment is derived from a biological sample and provides a template for the methods described herein, which interrogate nucleic acid sequences for the presence of a particular modified nucleobase. In this non-limiting, exemplary depiction, the modified nucleobase of interest is methylated cytosine (5-mC) and each strand of the DNA target fragment (i.e., sense strand “+” and antisense strand “−”) includes one 5-mC residue.

In some instances, the DNA sample is genomic DNA, mitochondrial DNA, cell free DNA (cfDNA), circulating tumor DNA (ctDNA), or a combination thereof, obtained from a biological sample.

The methods may then involve ligating adaptors to the ends of the double stranded DNA target fragments to produce adaptor-ligated DNA target fragments (step B). The adaptors may include a region of double stranded DNA and a region of single stranded DNA. In the example illustrated in FIG. 4A, the adaptors included two regions of single stranded DNA. In other embodiments, the adapters may have any suitable configuration for the downstream steps of a particular workflow. For example, in one embodiment, one of the adapters may be a hairpin adapter. The adaptors may also include sequences or other features that mediate downstream steps of the workflow, for example, sequences for immobilization of the adaptor-ligated DNA target fragments on a solid support, sequences for hybridization of oligonucleotide primer(s), sequences enabling bioinformatic analysis of DNA sequence information (e.g., unique molecular identifiers [UMI]s), and the like.

The methods may then include the step of denaturing the adaptor-ligated double stranded DNA target fragments to produce a single stranded target fragment sense (+) strand and a single stranded target fragment antisense (−) strand (step C).

The method may then include the step of performing a primer extension reaction. The primer extension reaction is directed by an extension oligonucleotide (i.e., an oligonucleotide primer), hybridized to the single stranded DNA target fragments using a DNA polymerase. The primer extension reaction produces a sample of double stranded DNA fragments, each including a complementary copy strand hybridized to a DNA target fragment strand (step D). In some instances, the DNA polymerase is a high-fidelity DNA polymerase. In this step, the sample of double stranded DNA fragments is distinguished from the sample of DNA target fragments of step 1A in that the former includes a complementary copy strand that is synthesized in vitro, while the later includes two target strands, each derived from the biological sample. The primer extension reaction is carried out under conditions in which the complementary copy strands produced are “native” strands, e.g., they do not include the modified nucleobases of interest present in the target strands. For example, in this depiction, the first complementary copy strands incorporate native cytosine residues at the positions of methylated cytosine residues in the target strands.

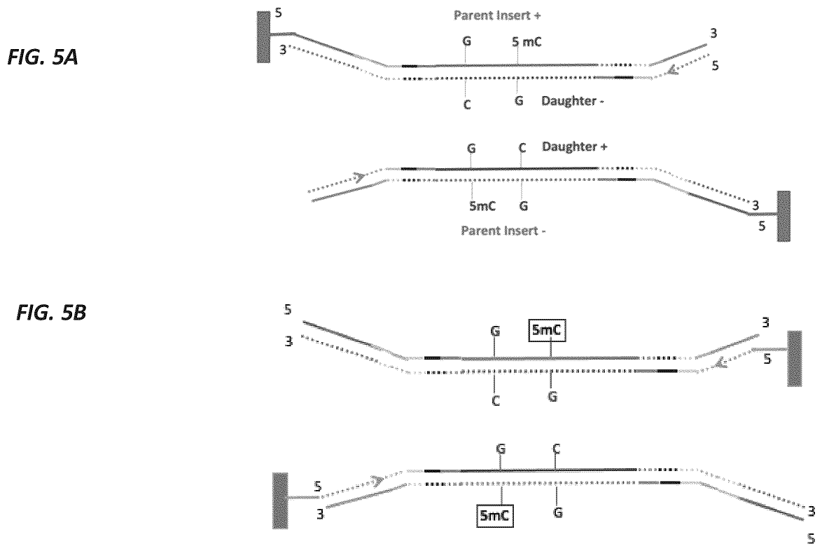

In some instances, the single stranded target fragments are immobilized on a solid support prior to the step of performing the first primer extension reaction, as depicted in FIG. 5A. As illustrated here, the complementary copy strands (i.e., the daughter strands) are not immobilized on the solid support and may be physically separated from the immobilized “parent” strands upon denaturation of the double stranded DNA fragments. In other instances, as depicted in FIG. 5B, an oligonucleotide complementary to the single stranded adaptor-ligated target fragment is immobilized on a solid support and is capable of “capturing” the single stranded target fragment on the solid support. Following capture of the target fragment, the first primer extension reaction may be performed to produce the first complementary copy strand immobilized on the solid support. In this instance, denaturation of the double stranded DNA fragment will release the single stranded target fragment from the solid support.

The methods may then involve treating the sample of double stranded DNA fragments with a DNA glycosylase enzyme that excises the nucleotides comprising the modified nucleobase of interest (e.g., 5-mC in this depiction). It is well-recognized in the art that glycosylase enzymes may be also classified as base-excision repair enzymes and both classes of enzymes may be used for the practice of methods disclosed herein. Excision of the nucleotides comprising the modified nucleobases of interest (i.e., modified nucleotides of interest) produces a single stranded, single nucleotide gap in the DNA target fragment at each position of the modified nucleotide of interest (Step E in FIG. 4A and depicted in FIG. 4B). In some instances, more than one DNA glycosylase or other enzyme(s) may be used to generate the single stranded gaps. A suitable combination of enzymes will provide both base-specific glycosylase activity and lyase activity to completely excise the modified nucleotides of interest from the DNA target fragment strands. A non-limiting list of exemplary DNA glycosylase enzymes is set forth in Table 1. Of note, according to the present invention, the complementary copy strands remain resistant to DNA glycosylase treatment, such that the native nucleotides in the complementary copy strands are not converted to single stranded, single nucleotide gaps. As used herein, the term “converted”, when used in reference to a DNA target fragment, refers to a DNA target fragment or a portion thereof which has been treated under conditions sufficient to convert the modified nucleotides of interest to single stranded gaps.

The methods may then include the subsequent steps of repairing the single stranded gaps in the DNA target strands by either the gap-ligation or gap-fill methodologies, as described herein. In either case, gap repair produces contiguous DNA target fragment strands (i.e., contiguous template strands derived from the parental target fragment). The repaired DNA parent strands and unconverted daughter strands may then be optionally amplified by conventional PCR technologies.

The methods may then include the step of determining the nucleotide sequences of the DNA parent and daughter strands. Diverse sequencing platforms and methodologies are suitable for the practice of the present invention. In one preferred example, the sequencing method is the Sequencing by Expansion (SBX) protocol developed by the inventors, see, e.g., U.S. Pat. Nos. 7,939,259 and 10,301,345 and published application nos, WO2020/172,479 and WO2020/236,526, which are herein incorporated by reference in their entireties.

The methods may then include the step of bioinformatically analyzing the sequence data to compare the sequences of the parent and daughter strands to determine the positions of the modified nucleobases of interest in the DNA target fragments prior to enzymatic conversion. The daughter (complementary strand) is used as a reference sequence as it encodes the genetic information of the original DNA target fragment. The parent strand encodes the epigenetic information of the DNA target fragment. Differences (e.g., the presence of a mutation in the sequence of the parent strand) in the sequences of the parent and daughter strands at a specific position indicate the position of the modified nucleobase of interest in the DNA target fragment. As disclosed herein, the gap-ligation protocol will yield deletions in the sequence of the parent strand relative to the daughter strand at positions of the modified nucleotide of interest, while the gap-fill protocol will yield base substitutions at the same positions.

Accordingly, in an embodiment of the method of the present invention, the step of comparing the nucleotide sequences of the DNA target fragments and the complementary copy strands identifies differences in the sequence of the DNA target fragments strands relative to the sequence of the_complementary copy strands, in which the positions of the one or more differences identifies the positions of the modified nucleobase base of interest in the DNA target fragments. In a certain embodiment, the differences are mutations, such as one or more mutations. In another embodiment, the differences are deletions, such as one or more deletions. In another embodiment, the differences are substitutions, such as one or more substitutions. In another embodiment, the differences are mutations, deletions and/or substitutions.

The methods provided herein are particularly useful in multiplex formats in which large numbers of DNA target fragments having different sequences and/or different nucleobase modification patterns are assayed in a common sample or pool. Thus, the methods set forth herein can provide the advantage of avoiding the need for separation of different target fragments into separate vessels during one or more steps of a nucleobase modification detection assay.

Further details regarding the above-described methods are provided below.

The practice of the present invention will employ, unless otherwise indicated, conventional techniques of molecular biology, microbiology, recombinant DNA, and so forth which are within the skill of the art. Such techniques are explained fully in the literature. See e.g., Sambrook, Fritsch, and Maniatis, MOLECULAR CLONING: A LABORATORY MANUAL, Second Edition (1989), OLIGONUCLEOTIDE SYNTHESIS (M. J. Gait Ed., 1984), the series METHODS IN ENZYMOLOGY (Academic Press, Inc.), CURRENT PROTOCOLS IN MOLECULAR BIOLOGY (F. M. Ausubel, R. Brent, R. E. Kingston, D. D. Moore, J. G. Siedman, J. A. Smith, and K. Struhl, eds., 1987).

### DNA Sample/Target Fragments

In one aspect, DNA from a biological sample is obtained or provided. The DNA obtained or provided from the biological sample may be genomic DNA, mitochondrial DNA, cell-free DNA (cfDNA), circulating tumor DNA (ctDNA), or a combination thereof.

DNA samples may be obtained from a patient or subject, from an environmental sample, or from an organism of interest. In some embodiments, the DNA sample is extracted, purified, or derived from a cell or collection of cells, a body fluid, a tissue sample, an organ, and/or an organelle. In preferred embodiments, the sample DNA is whole genomic DNA.

In some instances, genomic DNA and mitochondrial DNA may be obtained separately from the same biological sample or source. Many different methods and technologies are available for the isolation of genomic DNA and mitochondrial DNA. In general, such methods involve disruption and lysis of the starting material followed by the removal of proteins and other contaminants and finally recovery of the DNA. Removal of proteins can be achieved, for example, by digestion with proteinase K, followed by salting-out, organic extraction, gradient separation, or binding of the DNA to a solid-phase support (either anion-exchange or silica technology). Mitochondrial DNA may be isolated similarly following initial isolation of mitochondria. DNA may be recovered by precipitation using ethanol or isopropanol. There are also commercial kits available for the isolation of nuclear or mitochondrial DNA. The choice of a method depends on many factors including, for example, the amount of sample, the required quantity and molecular weight of the DNA, the purity required for downstream applications, and the time and expense.

The methods of the present disclosure utilize mild enzymatic and chemical reactions that avoid the substantial degradation associated with methods like bisulfite sequencing. Thus, the methods are useful in analysis of low-input samples, such as circulating cell-free DNA, circulating tumor DNA, and in single-cell analysis.

In some embodiments, the DNA sample is circulating cell-free DNA (cfDNA), which is DNA found in the blood and is not present within a cell. cfDNA can be isolated from blood or plasma using methods known in the art. Commercial kits are available for isolation of cfDNA including, for example, the Circulating DNA Kit (Qiagen). The DNA sample may result from an enrichment step, including, but is not limited to antibody immunoprecipitation, chromatin immunoprecipitation, restriction enzyme digestion-based enrichment, hybridization-based enrichment, or chemical labeling-based enrichment.

In some instances, the isolated DNA is fragmented into a plurality of shorter double stranded DNA pieces. In general, fragmentation of DNA may be performed physically, or enzymatically.

For example, physical fragmentation may be performed by acoustic shearing, sonication, microwave irradiation, or hydrodynamic shear. Acoustic shearing and sonication are the main physical methods used to shear DNA. For example, the Covaris® instrument (Woburn, MA) is an acoustic device for breaking DNA into 100 bp-5 kb. Covaris also manufactures tubes (gTubes) which will process samples in the 6-20 kb for Mate-Pair libraries. Another example is the Bioruptor® (Denville, NJ), a sonication device utilized for shearing chromatin, DNA and disrupting tissues. Small volumes of DNA can be sheared to 150 bp-1 kb in length. The Hydroshear® from Digilab (Marlborough, MA) is another example and utilizes hydrodynamic forces to shear DNA. Nebulizers, such as those manufactured by Life Technologies (Grand Island, NY) can also be used to atomize liquid using compressed air, shearing DNA into 100 bp-3 kb fragments in seconds. As nebulization may result in loss of sample, in some instances, it may not be a desirable fragmentation method for limited quantities samples. Sonication and acoustic shearing may be better fragmentation methods for smaller sample volumes because the entire amount of DNA from a sample may be retained more efficiently. Other physical fragmentation devices and methods that are known or developed can also be used.

Various enzymatic methods may also be used to fragment DNA. For example, DNA may be treated with DNase I, or a combination of maltose binding protein (MBP)-T7 Endo I and a non-specific nuclease such as Vibrio vulnificus nuclease (Vvn). The combination of non-specific nuclease and T7 Endo synergistically work to produce non-specific nicks and counter nicks, generating fragments that disassociate 8 nucleotides or less from the nick site. In another example, DNA may be treated with NEBNext® dsDNA Fragmentase® (NEB, Ipswich, MA). NEBNext® dsDNA Fragmentase generates dsDNA breaks in a time-dependent manner to yield 50-1,000 bp DNA fragments depending on reaction time. NEBNext dsDNA Fragmentase contains two enzymes, one randomly generates nicks on dsDNA and the other recognizes the nicked site and cuts the opposite DNA strand across from the nick, producing dsDNA breaks. The resulting DNA fragments contain short overhangs, 5 ‘-phosphates, and 3’-hydroxyl groups.

In some instances, the DNA sample is fragmented into specific size ranges. For example, the DNA sample may be fragmented into fragments in the range of about 25-100 bp, about 25-150 bp, about 50-200 bp, about 25-200 bp, about 50-250 bp, about 25-250 bp, about 50-300 bp, about 25-300 bp, about 50-500 bp, about 25-500 bp, about 150-250 bp, about 100-500 bp, about 200-800 bp, about 500-1300 bp, about 750-2500 bp, about 1000-2800 bp, about 500-3000 bp, about 800-5000 bp, or any other size range within these ranges. For example, the DNA sample may be fragmented into fragments of about 50-250 bp. In some instances, the fragments may be larger or smaller by about 25 bp.

The DNA target fragments may be any DNA fragment, derived from a biological sample, having a sequence of interest that may or may not include epigenetic modifications or DNA damage to one or more nucleobases. In some aspects, the DNA target fragments may include cytosine modifications (i.e., 5mC, 5hmC, 5fC, and/or 5caC). The DNA target fragments can be a single DNA molecule in the sample, or may be the entire population of DNA molecules in a sample (or a subset thereof) having, e.g., a cytosine modification. The DNA target fragments can comprise a plurality of DNA sequences such that the methods described herein may be used to generate a library of DNA target fragments that can be analyzed individually (e.g., by determining the sequence of individual targets) or in a group (e.g., by multiplexed DNA sequencing methodologies).

In embodiments, the methods described herein include the step of adding adaptor DNA molecules to double stranded DNA target fragments. An adaptor DNA, or DNA linker, is a short, chemically-synthesized, single- or double-stranded oligonucleotide that can be ligated to one or both ends of other DNA molecules. Double-stranded adaptors can be synthesized so that each end of the adaptor has a blunt end or a 5′ or 3′ overhang (i.e., sticky ends). DNA adaptors are ligated to the DNA target fragments to provide sequences for, e.g., primer extension reactions and sequencing reactions with complimentary primers and/or for bioinformatic analysis (e.g., clustering of related sequences into families based on shared unique molecular identifiers, UMIs).

Prior to ligation of adapters, the ends of the DNA fragments can be prepared for ligation. For example, by end repair and creating blunt ends with 5′ phosphate groups. Fragmented DNA may be rendered blunt-ended by a number of methods known to those skilled in the art. In a particular method, the ends of the fragmented DNA are “polished” with T4 DNA polymerase and Klenow polymerase, a procedure well known to skilled practitioners, and then phosphorylated with a polynucleotide kinase enzyme. A single ‘A’ deoxynucleotide is then added to both 3′ ends of the DNA molecules using Taq polymerase or Klenow exo minus polymerase enzyme, producing a one-base 3′ overhang that is complementary to the one-base 3′ ‘T’ overhang on the double-stranded end of an adaptor.

In some instances, the adaptors may include two oligonucleotides that are partially complementary such that they hybridize to form a region of double stranded sequence, but also retain a region of single stranded, non-hybridized sequence. The region of single stranded sequence may include “universal” oligonucleotide binding sequences, enabling all target fragments in a library to bind to the same oligonucleotide, which may be a capture oligonucleotide, to localize target fragments to a solid-support, an oligonucleotide primer for a primer extension reaction, a PCR primer, sequencing primer, or combinations thereof. In certain instances, the adaptors may include two regions of single-stranded, non-hybridized sequence (i.e., a first, 5′ single stranded region and a second, 3′ single stranded region). This configuration is known in the art as a “Y” adaptor. The first and second single stranded regions of a Y adaptor are not complementary and may include different primer hybridization sequences and other features.

The portions of the two single stranded regions of the adaptors typically include at least 10, or at least 15, or at least 20 consecutive nucleotides on each strand. The lower limit on the length of the single stranded regions will typically be determined by function, for example, the need to provide a suitable sequence for binding of a primer for primer extension, PCR and/or sequencing. Theoretically there is no upper limit on the length of the single stranded regions, except that in general it is advantageous to minimize the overall length of the adaptor, for example, in order to facilitate separation of unbound adaptors from adaptor-ligated double stranded DNA target fragments following the ligation step. Therefore, it is preferred that the single stranded regions should be fewer than 50, or fewer than 40, or fewer than 30, or fewer than 25 consecutive nucleotides in length on each strand.

The double stranded regions of the adaptor is a short double stranded region, typically comprising 5 or more consecutive base pairs, formed by annealing of the two partially complementary polynucleotide strands. Generally, it is advantageous for the double stranded region to be as short as possible without loss of function. By “function” in this context is meant that the double stranded region forms a stable duplex under standard reaction conditions for the enzyme-catalyzed nucleic acid ligation reaction.

The precise nucleotide sequence of the adaptors is generally not material to the invention and may be selected by the user such that the desired sequence elements are ultimately included in the common sequences of the library of adaptor-ligated double stranded DNA target fragments. Additional sequence elements may be included, for example, to provide binding sites for primers which will ultimately be used in sequencing of complementary copy strands of the DNA target fragments. The adaptors may further include “tag” sequences, unique molecular identifiers (UMI), and/or sample identifier sequences, which can be used to tag, track, and differentiate target fragments and complementary copies thereof derived from a particular source. The general features and use of such sequences is well known in the art.

The ends of the single stranded regions of the adaptors may be biotinylated or bear another functionalities that enables it to be captured, or immobilized, on a surface, such as a solid support. Alternative functionalities other than biotin are known to those skilled in the art and described in Applicant's published patent application no. WO2020/172479 entitled, “Methods and Devices for Solid-Phase Synthesis of Xpandomers for use in Single Molecule Sequencing”, which is herein incorporated by reference in its entirety.

“Ligation” of adaptors to the 5′ and 3′ ends of each fragmented double stranded nucleic acid target fragment involves joining of the two polynucleotide strands of the adaptor to the double-stranded target polynucleotide such that covalent linkages are formed between both strands of the two double-stranded molecules. Preferably such covalent linking takes place by formation of a phosphodiester linkage between the two polynucleotide strands but other means of covalent linkage (e.g., non-phosphodiester backbone linkages) may be used. However, it is an essential requirement that the covalent linkages formed in the ligation reactions allow for read-through of a polymerase, such that the resultant construct can be copied in a primer extension reaction using primers which bind to sequences in the regions of the adaptor-target construct that are derived from the adaptor molecules.

In some instances, the adaptors and DNA target fragments may be incubated with a ligase to covalently link the adaptors and DNA target fragments. Ligase catalyzes the formation of a phosphodiester bond between juxtaposed 5′ phosphate and 3′ hydroxyl termini in duplex DNA or RNA. The enzyme will join blunt end and cohesive end termini as well as repair single stranded nicks in duplex DNA. An exemplary ligase is T4 ligase, which is the most frequently used enzyme for cloning. Another ligase that may be used is E. coli DNA ligase, which preferentially connects cohesive double-stranded DNA end but is also active on blunt ends DNA in the presence of Ficoll or polyethylene glycol. Another ligase that may be used is DNA ligase Ilia, which is known to function in mitochondria.

The products of the ligation reaction may be subjected to purification steps in order to remove unbound adaptor molecules before the adaptor-target constructs are processed further.

The ligation of adaptors to both ends of the double stranded DNA target fragments gives rise to a pool of adaptor-ligated double stranded DNA target fragments with adaptors at both ends of the target.

There are several standard methods for separating the strands of an adaptor-ligated double stranded DNA target fragment by denaturation, including thermal denaturation, or chemical denaturation in either 100 mM sodium hydroxide solution or formamide solution. The pH of a solution of single-stranded DNA fragments can be neutralized by adjusting with an appropriate solution of acid, or preferably by buffer-exchange through a size-exclusion chromatography column pre-equilibrated in a buffered solution.

### Complementary Copy Strand (i.e., “Daughter” Strand)

In embodiments disclosed herein, a single stranded DNA target fragment provides a template nucleic acid (i.e., a “parent” strand) for the generation of a complementary copy strand (i.e., a “daughter” strand) of the target fragment via a primer extension reaction. As used herein, the term “primer extension reaction” is used interchangeably with “nucleic acid polymerization reaction” and refers to an in vitro method for making a new strand of nucleic acid or elongating an existing nucleic acid (e.g., DNA or RNA) in a template-dependent manner. The first complementary copy strand is generated by extending an oligonucleotide primer with a first DNA polymerase, such that a first complementary copy of the template strand is extended in the 3′ direction of the oligonucleotide primer.

In embodiments where the DNA target fragment is double stranded, one or both strands may serve as the template strand for the primer extension reactions. For example, where one strand (the “sense” strand) serves as template, a complementary copy is generated which is complementary to the sense strand. Likewise, where the antisense strand serves as template, a complementary copy is generated which is complementary to the antisense strand. Where both strands serve as template, a separate complementary copy is generated for each of the sense and antisense strands. In a preferred embodiment, each strand of a double stranded DNA target fragment is a template nucleic acid.

As used herein, the term “complementary” refers to nucleic acid sequences that are capable of forming Watson-Crick base-pairs. For example, a complementary sequence of a first sequence is a sequence which is capable of forming Watson-Crick base-pairs with the first sequence. The term “complementary” does not necessarily mean that a sequence is complementary to the full-length of its complementary strand, but the term can mean that the sequence is complementary to a portion thereof. Thus, in some embodiments, complementarity encompasses sequences that are complementary along the entire length of the sequence or a portion thereof. For example, two sequences can be complementary to each other along at least 70%, 75%, 80%, 85%, 90%, 91%, 92%, 93%, 94%, 95%, 96%, 97%, 98%, 99%, or 100% of the length of the sequence. Here, the term “sequence” encompasses, but is not limited to, nucleic acid sequences, polynucleotides, oligonucleotides, probes, primers, primer-specific regions, and target-specific regions. Despite the mismatches, the two sequences should have the ability to selectively hybridize to one another under appropriate conditions.

Primer extension can be performed by any method that allows for polymerase-based extension of a primer annealed (i.e., hybridized) to the single stranded DNA target fragment. In some embodiments, simple primer extension involves addition of a primer and a first DNA polymerase to the target DNA fragment under conditions to allow for primer hybridization and primer extension by the polymerase. Of course, such a reaction includes the necessary nucleotides, buffers, and other reagents known in the art for primer extension. Importantly, the nucleotides included in the primer extension reaction are “native”, i.e., unmodified, nucleotides and, thus, the complementary copy strand will not include modifications to the nucleobase of interest. The complementary copy strand is generated to encode and preserve the genetic sequence of the DNA target strand.

Any number of methods are known for detecting primer extension products. In some embodiments, the primer is detectably labeled (e.g., at its 5′ end or otherwise located to not interfere with 3′ extension of the primer) and following primer extension, the length and/or quantity of the labeled extension product is detected by detecting the label.

In a particular embodiment, the primer used in the primer extension reaction anneals to a primer-binding sequence (in one strand) in a single stranded region of the adaptor. The term “annealing” as used in this context refers to sequence-specific binding/hybridization of the primer to a primer-binding sequence in an adaptor region of the adaptor-ligated DNA target fragment under the conditions used for the primer annealing step of the initial primer extension reaction. Primer annealing conditions are well known in the art (see, e.g., Sambrook et al., 2001, Molecular Cloning, A Laboratory Manual, 3rd Ed, Cold Spring Harbor Laboratory Press, Cold Spring Harbor Laboratory Press, NY; Current Protocols, eds Ausubel et al.).

In preferred embodiments, the DNA polymerase is a high-fidelity DNA polymerase. The fidelity of a DNA polymerase is the result of accurate replication of a desired template. Specifically, this involves multiple steps, including the ability to read a template strand, select the appropriate nucleoside triphosphate and insert the correct nucleotide at the 3′primer terminus, such that Watson-Crick base pairing is maintained. In addition to effective discrimination of correct versus incorrect nucleotide incorporation, some DNA polymerases possess a 3′->5′ exonuclease activity. This activity, known as “proofreading”, is used to excise incorrectly incorporated mononucleotides that are then replaced with the correct nucleotide.

In certain embodiments, suitable high-fidelity DNA polymerases for the practice of the present invention include KAPA HiFi DNA Polymerase, commercially available from Roche Diagnostics Corp., Q5® High-Fidelity DNA Polymerase, commercially available from New England Biolabs, Inc., and an engineered Pfu DNA polymerase, such as Pfu-X, commercially available from Jena Biosciences.

### Solid-Phase Synthesis

In certain embodiments, the primer extension reaction may be conducted on a solid support. Thus, in further aspects, the invention provides a method for solid-phase nucleic acid synthesis using adaptor-ligated DNA target fragments, which have known sequences at their 5′ and 3′ ends (e.g., sequence features that have been designed into the adapters).

The terms “solid support”, “solid-state”, “solid-phase”, and “substrate” are used herein interchangeably and refer to a material or group of materials having a rigid or semi-rigid surface or surfaces. In many embodiments, at least one surface of the solid support will be substantially flat, e.g., a surface of a polymeric microfluidic card or chip. In some embodiments it may be desirable to physically separate regions of a card or chip for different reactions with, for example, etched channels, trenches, wells, raised regions, pins, or the like. According to other embodiments, the solid support(s) will take the form of insoluble beads, resins, gels, membranes, microspheres, or other geometric configurations composed of, e.g., controlled pore glass (CPG) and/or polystyrene.

The invention encompasses solid-phase synthesis methods in which a capture moiety is immobilized on a solid support. In certain instances, the capture moiety includes a first end covalently bound to the solid support and a second end that provides a functional group capable of binding to the 5′ end of a single stranded adapter-ligated DNA target fragment. In this case, the single stranded DNA target fragment is immobilized on the solid support, while the complementary copy strand is not immobilized on the solid support. In other instances, the capture moiety includes an extension oligonucleotide that is capable of hybridizing to the 3′ end of the single stranded adapter-ligated target fragment. The single stranded adapter-ligated DNA target fragment is hybridized to the extension oligonucleotide and a primer extension reaction is carried out. In this case, only the complementary copy strand is immobilized on the solid support. These alternative solid-phase synthesis configurations are illustrated in FIG. 5A and FIG. 5B.

The term “immobilized”, as used herein, refers to the association, attachment, or binding between a molecule (e.g., linker, adapter, or oligonucleotide) and a support in a manner that provides a stable association under the conditions of elongation, amplification, ligation, and other processes as described herein. Such binding can be covalent or non-covalent. Non-covalent binding includes electrostatic, hydrophilic and hydrophobic interactions. Covalent binding is the formation of covalent bonds that are characterized by sharing of pairs of electrons between atoms. Such covalent binding can be directly between the molecule and the support or can be formed by a cross linker or by inclusion of a specific reactive group on either the support or the molecule or both. Covalent attachment of a molecule can be achieved using a binding partner, such as avidin or streptavidin, immobilized to the support and the non-covalent binding of the biotinylated molecule to the avidin or streptavidin. Immobilization may also involve a combination of covalent and non-covalent interactions.

Any suitable covalent attachment means known in the art may be used for these purposes. The chosen attachment chemistry will depend on the nature of the solid support and any derivatization or functionalities applied thereto. The extension oligonucleotide may include a moiety, which may be a non-nucleotide chemical modification, to facilitate attachment. Certain exemplary embodiments of suitable surface chemistries include conventional streptavidin/biotin interaction chemistry and involve functionalization of a solid support, e.g., with a linker moiety that includes terminal a biotin moiety. In this embodiment, the 5′ end of single stranded DNA fragment (or oligonucleotide) is bound to the linker moiety. Attachment is mediated by a streptavidin moiety provided by the 5′ end of the single stranded DNA fragment. The linker moieties disclosed herein may be of sufficient length to connect the single stranded DNA fragment to the support such that the support does not significantly interfere with primer extension reaction.

Alternatively, immobilization of a capture moiety or oligonucleotide (e.g., an extension oligonucleotide) to a solid support may be accomplished by covalent linkage of the capture oligonucleotide to the solid support via a click reaction. In this embodiment, the covalent linkage may be mediated by a maleimide-PEG-alkyne linker that is crosslinked to the solid support. An alkyne moiety provided by the end of the linker distal to the substrate is capable of reacting with an azide group provided by the 5′ end of the capture oligonucleotide. Methods of functionalizing a solid support with maleimide-linker polymers is provided in Applicant's published patent application no. WO2020/172479, entitled, “Methods and Devices for Solid-Phase Synthesis of Xpandomers for use in Single Molecule Sequencing”, which is herein incorporated by reference in its entirety.

In certain instances, the linkage between the capture moiety and the solid support is cleavable, enabling primer extension products to be released from the support following synthesis. Cleavable linkers and methods of cleaving such linkers are known and can be employed in the provided methods using the knowledge of those of skill in the art. For example, the cleavable linker can be cleaved by an enzyme, a catalyst, a chemical compound, temperature, electromagnetic radiation or light. Optionally, the cleavable linker includes a moiety hydrolysable by beta-elimination, a moiety cleavable by acid hydrolysis, an enzymatically cleavable moiety, or a photo-cleavable moiety. In some embodiments, a suitable cleavable moiety is a photocleavable (PC) spacer or linker phosphoramidite available from Glen Research.

### Glycosylase-Mediated Excision of Modified Nucleotides

In one aspect, the methods of the present invention include the step of incubating the double stranded DNA fragment products of the primer extension reaction with a DNA glycosylase enzyme to specifically excise the modified nucleotides of interest. Many DNA glycosylases have been identified, targeting a wide range of specific modified nucleobases and DNA damage elements, including sequence mismatches and a large range of epigenetic modifications. Exemplary genetic modifications detectable by the described methods include, but are not limited to, 5-methylcytosine (5-mC), 5-hydroxymethylcytosine (5-hmC), 5-carboxycytosine (5-caC), f5-ormylcytosine (5-fC), 8-oxo-7,8-dihyroguanine (oxoG), uracil, methyladenine (mA), and others.

There are two main classes of DNA glycosylases: monofunctional and bifunctional. Monofunctional glycosylases have only glycosylase activity and cleave the N-glycosidic bond linking a damaged or modified nucleobase to the sugar-phosphate backbone of DNA. All DNA glycosylases cleave glycosidic bonds, but differ in their base substrate specificity and in their reaction mechanisms. Bifunctional glycosylases also possess apurinic or apyrimidinic site (AP) lyase activity that permits them to cut the phosphodiester bond of DNA at a base lesion, creating a single-strand break. The methods disclosed herein require that the DNA glycosylase or combination of glycosylases provide both glycosylase and lyase activities in order to completely excise the modified nucleotides of interest from the DNA target strand.

Exemplary DNA glycosylases that may find use in the described methods are listed in Table 1. In some instances, one or more of DNA glycosylases listed in Table 1 may be used in the described methods to excise modified nucleotides of interest from DNA target fragments. While select DNA glycosylases are specifically identified in this disclosure, it is understood that any DNA glycosylase can be used in the performing the excision step of the described methods.

TABLE 1
DNA Glycosylases

| Enzyme | Type | Species | Substrate(s) |
|---|---|---|---|
| N-methylpurine | Monofunctional | Human | 3-mA; 7-mG; 3-mG |
| DNA | DNA | | |
| Glycosylase | glycosylase | | |
| (MPG) | | | |
| MutY Homolog | Monofunctional | Human | A mis-paired with 8-oxoG, C, |
| (MUTYH) | DNA | | or G |
| | glycosylase | | |
| Nth-like DNA | Bifunctional | Human | Thymine glycol (Tg); 2,6- |
| Glycosylase 1 | DNA | | diamino-4-oxo-5- |
| (NTHL1) | Glycosylase | | formamidopyrimidine (FapyG); |
| | | | 5-hmC; 5-hydroxyuracil (5- |
| | | | HU) |
| Nei-like DNA | Bifunctional | Human | Tg; FapyG; 4,6-diamino-5- |
| Glycosylase 1 | DNA | | formamidopyrimidine (FapyA); |
| (NEIL1) | Glycosylase | | 8-oxoG; 5-HU |
| NEIL2 | Bifunctional | Human | Tg; FapyG; FapyA; 8-oxoG; 5- |
| | DNA | | HU |
| | Glycosylase | | |
| NEIL3 | Mono- or | Human | FapyG, FapyA, |
| | bifunctional | | |
| | DNA | | |
| | glycosylase | | |
| 8-oxoguanine | Mono- or | Human | 8-oxoG |
| DNA | bifunctional | | |
| glycosylase | DNA | | |
| (OGG1) | glycosylase | | |
| Uracil DNA | Monofunctional | Human | U, 5-FU |
| Glycosylase 1 | DNA | | |
| (Ung1) | glycosylase | | |
| Uracil DNA | Monofunctional | Human | U, 5-FU |
| Glycosylase 2 | DNA | | |
| (Ung2) | glycosylase | | |
| Single-strand | Monofunctional | Human | 5-hmU; U mis-paired with G; |
| selective | DNA | | 5-FU |
| monofunctional | glycosylase | | |
| uracil | | | |
| glycosylase | | | |
| (SMUG1) | | | |
| Thymine DNA | Monofunctional | Human | U or T mis-paired with G; 5- |
| Glycosylase | DNA | | hmU; 5-fC, 5-CaC |
| (TDG) | glycosylase | | |
| Methyl binding | Monofunctional | Human | U or T mis-paired with G; 5- |
| domain 4 | DNA | | hmU in CpG islands |
| (MBD4) | glycosylase | | |
| Fpg | Bifunctional | E. coli | 8-oxoG; 5-caC |
| | DNA | | |
| | Glycosylase | | |
| Ung | Monofunctional | E. coli | Uracil |
| | DNA | | |
| | glycosylase | | |
| Demeter | Bifunctional | A. | 5-mC |
| | DNA | thaliana | |
| | Glycosylase | | |
| ROS1 | Bifunctional | A. | 5-mC; T mis-paired with G |
| | DNA | thaliana | |
| | Glycosylase | | |
| Apurinic | AP | Human | AP sites |
| endonuclease | endonuclease | | |
| (APE1) | | | |
| DNA | DNA | Human | Gaps in DNA |
| polymerase beta | polymerase | | |
| (POLB) | | | |
| XRCC1 | scaffold protein | Human | Stabilizes Lig3 |
| DNA Ligase 1 | DNA ligase | Human | DNA nick |
| (LIG1) | | | |
| DNA Ligase 3 | DNA ligase | Human | DNA nick (with XRCC1) |
| (LIG3) | | | |
| DNA | DNA | Human | Gaps in DNA (mitochondria) |
| polymerase | polymerase | | |
| gamma (POLG) | | | |

In one instance, a suitable DNA glycosylase that directly excises 5mC may be a member of the DEMETER (DME) family of DNA glycosylases, e.g., DME, ROS1, and DEMETER-like protein 2 (DMEL-2, DML2) and DEMETER-like protein 3 (DMEL-3, DML3). The DME gene of Arabidopsis encodes a 1,729 amino acid protein with a centrally located DNA glycosylase domain (amino acids 1167-1368) that includes a helix-hairpin-helix (HhH) motif. The HhH motif in DME catalyzes excision of 5-methylcytosine (see, e.g., Choi et al., 2002. Cell 110:33-42).

In some instances, a suitable DNA glycosylase that acts directly on 5mC may be an orthologue of DME. As used herein, the term “orthologue” means one of two or more homologous gene sequences found in different species. Table 2 sets forth an exemplary list of DME orthologues that may be used according to the present invention.

TABLE 2
DME Orthologues

| Species | Accession number/comments |
|---|---|
| Bathycoccus prasinos | XP_007509798 |
| Ostreococcus tauri | CEF97905 |
| Coccomyxa subellipsoidea | XP_00549215 |
| volvox carteri | XP_002950363 |
| Micromonas commode | XP_002500051 |
| Auxenochlorella protothecoides | XP_011399155 |
| Gonium pectoral | KXZ53817 |
| Physcomitrella patens | XP_001785533 |
| Physcomitrella patens | XP_001777735 |
| Nannochloropsis gaditana | EWM29049 |
| Klebsormidium nitens | GAQ84186 |
| Selaginella moellendorffii | XP_002990766 |
| Marchantia polymorpha | OAE23247 |
| N. gaditana | EJK65312 |
| Prunus avium | XP_021824383 |
| Prunus avium | XP_021833615 |
| Arabidopsis thaliana | OAO92434 (DME) |
| Capsicum annuum | XP_016550501 |
| Pyrus × bretschneideri | XP_009353006 |
| Capsicum annuum | XP_016543466 |
| Capsicum annuum | XP_016542890 |
| Arabidopsis thaliana | OAP06165 (DML2) |
| Arabidopsis thaliana | OAP10755 (ROS1) |
| Rubus occidentalis | GDR |
| Pyrus × bretschneideri | XP_009366838 |
| Malus domestica | XP_017178103 |
| Capsicum annuum | XP_016554458 |
| Capsicum annuum | Capsicum annuum_XP_016562450 |
| Arabidopsis thaliana | OAO99112 (DML3) |
| Capsicum annuum | XP_016566581 |
| Arabidopsis thaliana | NP_973767 (EndIII2) |
| Arabidopsis thaliana | NP_565725 (EndIII) |
| Arabidopsis thaliana | NP_566893 (DNAglycosylase protein) |
| Capsicum annuum | XP_016563498 |

In instances where the DNA glycosylase is a bifunctional enzyme, the DNA glycosylase (e.g., DME, or an orthologue thereof), exhibits both glycosylase and lyase activity. The reaction mechanism of bifunctional DNA glycosylases is well known in the art (see, e.g., Scharer and Jiricny. 2001. Bioessays 23:270-281). In some cases, a conserved aspartic acid acquires a proton from a conserved lysine residue that attacks the Cl′ carbon of the deoxyribose ring, creating a covalent DNA-enzyme intermediate. Beta or gamma elimination reactions release the enzyme from the DNA and cleave one of the phosphodiester bonds.

Mutations that inactivate or optimize suitable features of the DNA glycosylase are also contemplated by the present invention. For example, the DNA glycosylase may be engineered to increase its stability and/or solubility. The DNA glycosylase may also be engineered to optimize for a desired substrate specificity.

In one embodiment, thymine DNA glycosylase (TDG) may be used to excise its known targets of 5-carboxycytosine (5-caC) and 5-formylcytosine (5-fC) and, with additional steps of modifying bases in a DNA sample, may be used to identify 5-methylcytosine (5-mC) and 5-hydroxymethylcytosine (5-hmC), which are modified bases that it does not specifically recognize. For example, DNA target fragments may be treated with ten eleven translocation (TET) enzyme prior to treatment with TDG. The TET family proteins included three human proteins (TET1, TET2, and TET3) and are cytosine oxygenases that catalyze the conversion of 5-methylcytosine (5-mC) into 5-hydroxymethylcytosine (5-hmC). 5-hmC can be further oxidized into 5-formylcytosine (5-fC) and 5-carboxylcytosine (5-caC) by TET proteins (see, e.g., Parker, et. al. 2019. Biochemistry 58:450-467). In another instance, a suitable TET enzyme may be “nTET” (i.e., “ngTET”), isolated from Naegleria (see, e.g., Hashimoto, et. al. 2014. Nature 506 (7488): 391-395). TDG may be used to excise any existing 5-caC and -5fC modified bases present in the DNA target fragments.

Other comparable methods for altering the selective excision of modified nucleotides are possible. For example, a similar method may be performed to detect the same bases using thymine DNA glycosylase (TDG) and uracil DNA glycosylase (UDG).

The above-described nucleotide excision processes are performed using purified enzymes, which may be a recombinant enzyme including a heterologous tag to facilitate purification. Protein tags are well known in the art and include, e.g., terminal poly-histidine tags that enable purification via immobilized metal affinity chromatography (IMAC). In certain instances, it may be desirable to include more than one protein purification step. For example, the glycosylases enzymes used in the methods disclosed herein should preferably be free of contaminating nucleic acids. In some instances, the protein purification step includes one or more of size-exclusion chromatography, ion exchange chromatography, affinity chromatography, and the like.

In some instances, the nucleotide excision reaction includes a suitable buffer, suitable cofactors, additives, and an amount of purified DNA glycosylase sufficient to achieve the desired base excision reactions such that all modified nucleobases of interest in a DNA target fragment are excised to generate abasic sites.

Following treatment with the DNA glycosylase, the double stranded DNA fragment will be asymmetrically altered. Importantly, the DNA target strand (i.e., the parent strand) includes single stranded gaps at the original positions of the modified base of interest. In contrast, the complementary copy strand (i.e., the daughter strand) remains unaltered (i.e., “unconverted”), as the native nucleotides incorporated during the first primer extension reaction will be resistant to glycosylation-mediated conversion to single stranded gaps.

According to the present invention, the DNA target strands and complementary copy strands can be assessed through a number of established and emerging nucleic acid sequencing techniques, including, but not limited to, deep sequencing, next generation sequencing, and nanopore sequencing.

### Sequencing by Expansion

One preferred nanopore sequencing technique of the methods disclosed herein is the “Sequencing by Expansion” (SBX) protocol, developed by Stratos Genomics (see, e.g., Kokoris et al., U.S. Pat. No. 7,939,259, “High Throughput Nucleic Acid Sequencing by Expansion”). SBX is based on the polymerization of highly modified, non-natural nucleotide analogs referred to as “XNTPs”. In general terms, SBX uses biochemical polymerization to transcribe the sequence of a DNA template (e.g., the first and second complementary copy strands of the DNA target fragments) onto a measurable polymer called an “Xpandomer”. The transcribed sequence is encoded along the Xpandomer backbone in high signal-to-noise reporters that are separated by ˜10 nm and are designed for high-signal-to-noise, well-differentiated responses. These differences provide significant performance enhancements in sequence read efficiency and accuracy of Xpandomers relative to natural DNA.

XNTPs are expandable, 5′ triphosphate modified non-natural nucleotide analogs compatible with template dependent enzymatic polymerization. The XNTP has two distinct functional regions; namely, a selectively cleavable phosphoramidate bond, linking the 5′ α-phosphate to the nucleobase, and a symmetrically synthesized reporter tether (SSRT) that is attached within the nucleoside triphosphoramidate at positions that allow for controlled expansion by cleavage of the phosphoramidate bond. The SSRT includes linkers separated by the selectively cleavable phosphoramidate bond. Each linker attaches to one end of a reporter code. XNTP substrates and the those incorporated into daughter strand products of template-dependent polymerization are in the “constrained” configuration. The constrained configuration of polymerized XNTPs is the precursor to the expanded configuration, as found in Xpandomer products. The transition from the constrained configuration to the expanded configuration occurs upon scission of the P-N bond of the phosphoramidate within the primary backbone of the daughter strand.

The transition from the constrained configuration to an expanded configuration results from cleavage of the selectively cleavable phosphoramidate bonds within the primary backbone of the daughter strand. In this embodiment, the SSRTs include one or more reporters or reporter codes, specific for the nucleobase to which they are linked, thereby encoding the sequence information of the template. In this manner, the SSRTs provide a means to expand the length of the Xpandomer and lower the linear density of the sequence information of the parent strand.

The SSRT (i.e., “tether”) of the XNTP includes several functional elements, or “features” such as polymerase enhancement regions, the reporter codes, and translation control element (TCEs). Each of these features performs a unique function during translocation of the Xpandomer through a nanopore to produce a series of unique and reproducible electronic signal. The SSRT is designed for controlling the rate of Xpandomer translocation by the TCE through a combination of sterics and/or electrorepulsion. Different reporter codes are sized to block ion flow through a nanopore at different measurable levels. Specific SSRT polymeric sequences can be efficiently synthesized using phosphoramidite chemistry typically used for oligonucleotide synthesis. Reporter codes and other features can be designed by selecting a sequence of specific phosphoramidites from commercially available and/or proprietary libraries. Such libraries include, but are not limited to, polyethylene glycol with lengths of 1 to 12 or more ethylene glycol units and aliphatic polymers with lengths of 1 to 12 or more carbon units. In certain embodiments, the SSRTs include features referred to as “polymerase enhancement regions” at the ends of the SSRTs proximal to the nucleotide triphosphoramidate diester. Polymerase enhancement regions may include positively charged polyamine spacers (e.g., primary, secondary, tertiary, or quarternary amines) or triamine spacers (three secondary amines each separated by three carbons) that facilitate incorporation of XNTP structures by a nucleic acid polymerase. In certain embodiments, the polymerase enhancement region includes two repeat units spermine

As used throughout the present disclosure, the term “reporter construct” refers to the element of the SSRT that includes the reporter codes, a symmetrical chemical brancher, and a translocation control element. In certain embodiments, the reporter construct is a polymer that includes, in series, from a first end to a second end, a first reporter code, a symmetrical chemical brancher bearing a translocation control element, and a second reporter code. The term “bearing” refers to a covalent linkage between the symmetrical brancher and the translocation control element, which produces an advantageous orientation of the translocation control element with respect to the two reporter codes. As discussed further herein, the symmetrical chemical brancher can be represented by the letter “Y”, in which the two reporter codes are joined to the arms of the Y, while the translocation control element is joined to the stem of the Y. Thus, the two reporter codes are joined in-line by the brancher, while the brancher bears the translocation control element in a perpendicular orientation with respect to the linear, in-line, SSRT.

As used throughout the present disclosure, the terms “linker A” and “linker B” refer to the regions of the SSRT that each include a polymerase enhancing region and one or more translocation deceleration features or regions, and, in certain embodiments, a spacer region that includes a polymer of, e.g., PEG6, which can be customized to modulate the length of the SSRT traversed in a nanopore.

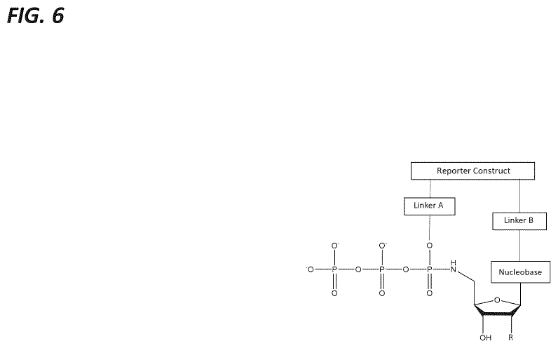

In certain embodiments, an XNTP may be a compound having the generalized structure as depicted in FIG. 6.

In one embodiment, R may be H, for example, when the compounds are used to sequence a DNA template.

In certain embodiments, nucleobase is adenine, cytosine, guanine, thymine, uracil or a nucleobase analog. As one of skill in the art will appreciate, adenine, cytosine, guanine, thymine, and uracil are naturally occurring nucleobases. As used herein, the term “nucleobase analog” refers to non-naturally occurring nucleobases that are capable of forming Watson and Crick base pair with a complementary nucleobase on an adjacent single-stranded nucleic acid template.

As discussed herein, the reporter construct is a polymer having a first end and a second end, and includes, in series from the first end to the second end, the first reporter code, the symmetrical chemical brancher bearing the translocation control element, and the second reporter code. This series of features reflects the symmetrical structure of the reporter construct (and the entire SSRT, which includes the symmetrical linkers, linker A and linker B), in which the sequences of the two reporter codes are identical and joined, in-line in reverse orientation by the symmetrical chemical brancher.

To obtain sequence information, an Xpandomer is translocated through a nanopore, from the cis reservoir to the trans reservoir. As the Xpandomer translocates, a reporter enters the stem until its translocation control element stops at the stem entrance. The reporter is held in the stem until the TCE is enabled to pass into and through the stem, whereupon translocation proceeds to the next reporter. Upon passage through the nanopore, each of the reporter codes of the linearized Xpandomer generates a distinct and reproducible electronic signal, specific for the nucleobase to which it is linked.

In certain embodiments, Xpandomers produced by the SBX chemistry may be analyzed using a nanopore-based sequencing chip. A nanopore-based sequencing chip can incorporate a large number of sensor cells configured as an array. For example, the chip may include an array of one million cells configured in 1000 rows by 1000 columns of cells. Each cell in the array may include a control circuit integrated on a silicon substrate. Such nanopore-based sequencing chips, devices, and systems are described, e.g., in Applicant's published patent application no. WO2021/219795, which is herein incorporated by reference in its entirety. Diagnostic and Prognostic Methods

In particular embodiments, the methods can be directed to diagnosing an individual with a condition that is characterized by a methylation level and/or pattern of methylation at particular loci in a test sample that are distinct from the methylation level and/or pattern of methylation for the same loci in a sample that is considered normal or for which the condition is considered to be absent. The methods can also be used for predicting the susceptibility of an individual to a condition that is characterized by a level and/or pattern of methylated loci that is distinct from the level and/or pattern of methylated loci exhibited in the absence of the condition.

Exemplary conditions that are suitable for analysis using the methods set forth herein can be, for example, cell proliferative disorder or predisposition to cell proliferative disorder; metabolic malfunction or disorder; immune malfunction, damage or disorder; CNS malfunction, damage or disease; symptoms of aggression or behavioral disturbance; clinical, psychological and social consequences of brain damage; psychotic disturbance and personality disorder; dementia or associated syndrome; cardiovascular disease, malfunction and damage; malfunction, damage or disease of the gastrointestinal tract; malfunction, damage or disease of the respiratory system; lesion, inflammation, infection, immunity and/or convalescence; malfunction, damage or disease of the body as an abnormality in the development process; malfunction, damage or disease of the skin, the muscles, the connective tissue or the bones; endocrine and metabolic malfunction, damage or disease; headache or sexual malfunction, and combinations thereof.

Abnormal methylation of CpG islands associated with tumor suppressor genes can cause decreased gene expression. Increased methylation of such regions can lead to progressive reduction of normal gene expression resulting in the selection of a population of cells having a selective growth advantage. Conversely, decreased methylation (hypomethylation) of oncogenes can lead to modulation of normal gene expression resulting in the selection of a population of cells having a selective growth advantage.

Accordingly, in particular embodiments a disease or condition to be analyzed with respect to methylation levels is cancer. Exemplary cancers that can be evaluated using a method of the invention include, but are not limited to cancer of the breast, prostate, lung, bronchus, colon, rectum, urinary bladder, kidney, renal pelvis, pancreas, oral cavity or pharynx (Head & Neck), ovary, thyroid, stomach, brain, esophagus, liver, intrahepatic bile duct, cervix, larynx, soft tissue such as heart, testis, gastro-intestinal stroma, pleura, small intestine, anus, anal canal and anorectum, vulva, gallbladder, bones, joints, hypopharynx, eye or orbit, nose, nasal cavity, middle ear, nasopharynx, ureter, peritoneum, omentum, or mesentery. Other cancers that can be evaluated include, for example, Chronic Myeloid Leukemia, Acute Lymphocytic Leukemia, Malignant Mesothelioma, Acute Myeloid Leukemia, Chronic Lymphocytic Leukemia, Multiple Myeloma, Gastrointestinal Carcinoid Tumors, Non-Hodgkin Lymphoma, Hodgkin Lymphoma or Melanomas of the skin.

With particular regard to cancer, changes in DNA methylation have been recognized as one of the most common molecular alterations in human neoplasia. Hypermethylation of CpG islands located in the promoter regions of tumor suppressor genes is a well-established and common mechanism for gene inactivation in cancer (Esteller, Oncogene 21(35): 5427-40 (2002)). In contrast, a global hypomethylation and increased gene expression has been reported for many oncogenes (Feinberg, Nature 301(5895): 89-92 (1983), Hanada, et al., Blood 82(6): 1820-8 (1993)). Cancer diagnosis or prognosis can be made in a method set forth herein based on the methylation state of particular sequence regions of a gene including, but not limited to, the coding sequence, the 5′-regulatory regions, or other regulatory regions that influence transcription efficiency.

A reference genomic DNA (for example, gDNA considered “normal”) and a test genomic DNA that are to be compared in a diagnostic or prognostic method, can be obtained from different individuals, from different tissues, and/or from different cell types. In particular embodiments, the genomic DNA samples to be compared can be from the same individual but from different tissues or different cell types, or from tissues or cell types that are differentially affected by a disease or condition. Similarly, the genomic DNA samples to be compared can be from the same tissue or the same cell type, wherein the cells or tissues are differentially affected by a disease or condition.

What is claimed is:

1. A method of detecting a modified nucleobase in a plurality of nucleic acids, the method comprising:
providing a sample comprising a plurality of DNA templates;
generating complementary copies of the DNA templates, the generating being directed by an oligonucleotide primer using a DNA polymerase in the presence of native dNTPs, wherein the generating produces a complementary copy of each of the DNA templates such that each complementary copy is hybridized to one of the DNA templates;
subjecting the DNA templates and the complementary copies to a base excision repair enzyme treatment, wherein the base excision repair enzyme specifically excises the nucleotides comprising the modified nucleobase from the DNA templates to produce a single stranded gap at the positions of the modified nucleobase, and wherein the complementary copies are resistant to treatment with the base excision repair enzyme;
repairing the single stranded gaps in the DNA templates to produce contiguous DNA template strands;
determining the nucleotide sequences of the contiguous DNA template strands and the complementary copies; and
comparing the nucleotide sequences of contiguous DNA template strands and the complementary copies, thereby determining the positions of the modified nucleobase in the DNA templates prior to base excision repair enzyme treatment.

2. The method of claim 1, wherein the step of repairing the single stranded gaps in the DNA templates to produce the contiguous DNA template strands comprises treating the DNA templates with a DNA ligase enzyme, thereby producing a deletion in the contiguous DNA template strands at each of the positions of the nucleotides comprising the modified nucleobase in the DNA templates.

3. The method of claim 1, wherein the step of repairing the single stranded gaps in the DNA templates to produce the contiguous DNA template strands comprises treating the DNA templates with a DNA polymerase enzyme and a DNA ligase enzyme in the presence of a non-native nucleotide, thereby producing a nucleotide substitution in the contiguous DNA template strands at each of the positions of the nucleotides comprising the modified nucleobase in the DNA templates.

4. The method claim 1, wherein the step of comparing the nucleotide sequences of the contiguous DNA template strands and the complementary copies identifies one or more differences in the sequence of the contiguous DNA template strands relative to the sequence of the complementary copies, wherein the positions of the differences identifies the positions of the modified nucleobase base in the DNA templates.

5. The method of claim 4, wherein the one or more differences in the sequence of the DNA target fragments strands relative to the sequence of the complementary copy strands are one or more mutations, one or more deletions, or one or more substitutions.

6. The method of claim 1, wherein the base excision repair enzyme is selected from N-methylpurine DNA Glycosylase (MPG), MutY Homolog (MUTYH), Nth-like DNA Glycosylase 1 (NTHL1), Nei-like DNA Glycosylase 1 (NEIL1), Nei-like DNA Glycosylase 2 (NEIL2), Nei-like DNA Glycosylase 3 (NEIL3), 8-oxoguanine DNA glycosylase (OGG1), Uracil DNA Glycosylase 1 (Ung1), Uracil DNA Glycosylase 2 (Ung2), Single-strand selective monofunctional uracil glycosylase (SMUG1), Thymine DNA Glycosylase (TDG), Methyl binding domain 4 (MBD4), FPG, Ung, Demeter (DME), DMEL-2, DMEL-3, ROS1, UDG, Apurinic endonuclease (APE1), DNA polymerase beta (POLB), XRCC1, DNA Ligase 1 (LIG1), DNA Ligase 3 (LIG3), and DNA polymerase gamma (POLG).

7. The method of claim 1, wherein the base excision repair enzyme comprises a DNA glycosylase enzyme, wherein the DNA glycosylase enzyme exhibits glycosylase activity and lyase activity.

8. The method of claim 6, wherein the DNA glycosylase enzyme is selected from the group consisting of FPG, DME, ROS1, DMEL-2, and DMEL-3.

9. The method of claim 1, wherein the base excision repair enzyme comprises a first enzyme exhibiting glycosylase activity and a second enzyme exhibiting lyase activity.

10. The method of claim 9, wherein the first enzyme is TDG or UDG and the second enzyme is selected from the group consisting of FPG, DME, ROS1, DMEL-2, and DMEL-3.

11. The method of claim 1, wherein the DNA polymerase is a high-fidelity DNA polymerase.

12. The method of claim 1, wherein the DNA templates comprise genomic DNA, mitochondrial DNA, cell-free DNA, circulating tumor DNA, or combinations thereof.

13. The method of claim 1, wherein the modified nucleobase is selected from the group consisting of 5-mC, 5-hmC, 5-fC, and 5-caC.

14. The method of claim 1, wherein the DNA templates are immobilized on a solid support.

15. The method of claim 1, wherein the complementary copies are immobilized on a solid support.

16. The method of claim 1, wherein the method further comprises the step of polishing the single stranded gaps with one or more enzymes to produce a free 3′ hydroxyl group and a free 5′ phosphate group at the positions of each of the gaps.

17. The method of claim 16, wherein the one or more enzymes is selected from the group consisting of APE1, Endonuclease B, PolB, and PNK.

18. The method of claim 3, wherein the non-native nucleotide is selected from the group consisting of dZTP, dPTP, dSTP, and dBTP.

19. The method of claim 3, wherein the DNA polymerase enzyme does not exhibit exonuclease activity or strand displacing activity and the DNA ligase enzyme is not capable of ligating across single stranded gaps.

20. The method of claim 19, wherein the DNA polymerase enzyme is Klenow exo- or T4 DNA polymerase and the DNA ligase enzyme is E. coli DNA ligase.

21. The method of claim 2, wherein the DNA ligase enzyme is T4 DNA ligase.

22. The method of claim 1, wherein the DNA templates comprise a first adapter joined to the 5′ end of the DNA template and a second adapter joined to the 3′ end of the DNA template.

23. The method of claim 22, wherein the first adapter is a Y adapter and the second adapter is a Y adapter or a hairpin adapter.

24. The method of claim 23, wherein at least one of the first and the second adapters comprises a unique molecular identifier barcode (UMI).

25. The method of claim 24, wherein the step of comparing the sequences of the contiguous DNA template strands and the complementary copies comprises bioinformatically pairing the sequences comprising the same unique molecular barcode (UMI).
